(19) United States
(12) Patent Application Publication
(10) Pub. No.: US 2014/0178346 A1
(43) Pub. Date: Jun. 26, 2014

(54) CELLULAR COMPOSITIONS FOR TISSUE ENGINEERING

(71) Applicant: THE REGENTS OF THE UNIVERSITY OF CALIFORNIA, Oakland, CA (US)

(72) Inventors: James A. Byrne, Santa Monica, CA (US); Jeffrey C. Wang, Sherman Oaks, CA (US)

(73) Assignee: THE REGENTS OF THE UNIVERSITY OF CALIFORNIA, Oakland, CA (US)

(21) Appl. No.: 14/140,420

(22) Filed: Dec. 24, 2013

Related U.S. Application Data
(60) Provisional application No. 61/746,025, filed on Dec. 26, 2012.

(51) Int. Cl. A61K 35/32

(52) U.S. Cl. CPC A61K 35/32
USPC 424/93.21

(57) ABSTRACT

Cell compositions for tissue engineering are provided which contain a population of autologous, minimally passaged dermal fibroblasts in combination with a tissue engineering matrix or scaffold, or material forming a matrix or scaffold. In one embodiment, the population of fibroblasts is genetically engineered to secrete a therapeutic protein in an amount effective to induce tissue growth or tissue repair when the cell composition is transplanted into a subject in need thereof. For example, the therapeutic protein can be a bone morphogenic protein when the tissue to be treated is bone tissue. A preferred bone morphogenic protein is BMP-2.

### CROSS-REFERENCE TO RELATED APPLICATIONS

This application claims benefit of and priority to U.S. provisional patent application Ser. No. 61/746,025 filed on Dec. 26, 2012, which is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED IN A COMPUTER PROGRAM APPENDIX

Not Applicable

### BACKGROUND OF THE INVENTION

1. Field of the Invention

The invention is generally directed to cellular compositions and methods for treating bone defects and disorders.

2. Background Discussion

Tissue engineering and regenerative medicine are providing exciting new treatments to help heal damaged organs and tissues. One important aspect of tissue engineering is the ability to use a person's own cells to treat that person. By using autologous cells, the risk of tissue rejection or graft rejection is eliminated. One of the fasted growing segments of tissue engineering is in the treatment of bone disorders and disease.

Spinal fusion is a routinely performed medical procedure in the United States, with over quarter of a million spinal fusions performed each year to treat severe lower back pain. The two common methods for performing spinal fusion are iliac crest autografting and non-cellular recombinant human BMP-2 (rh-BMP-2). Both approaches have their own set of problems.

Bone grafting is a reconstructive orthopedic surgical procedure that is used to provide structural support and augment bone regeneration in the treatment of a variety of conditions. The most common source of autologous bone for grafting is both the anterior and posterior portions of the iliac crest of a hip of the patient. Alternative sites such as the intramedullary canal of long bones can be a source of both cortical and/or cancellous bone for grafting. The harvested bone from the hip is used in a variety of reconstructions such as vertebral fusions, the repair of difficult fractures, bone defect replacements and the repair of non-unions.

Grafting with autologous bone is the gold standard and is preferred over the use of cadaver allografts, xenografts and synthetic bone substitutes because autologous bone does not create a risk of transmission of viral diseases or the induction of an immune response. However, hip bone autografting has been significantly associated with donor site morbidity, bone supply limitations and post-surgical pain as well as other complications. Major complications from harvesting include hematoma requiring surgical intervention, vascular or neurological injuries and deep infections at the donor site, iliac wing fractures, muscle destabilization and chronic pain.

Recombinant human BMP-2 (rhBMP-2) represents the main alternative to hip bone autografting and typically uses non-cellular delivery of recombinant human rhBMP-2 that has been shown to promote new bone formation. However, administration of recombinant human BMP-2 can result in a significant spike in the initial BMP-2 concentration far above the normal physiological concentrations that is believed to be associated with negative health consequences. Non-cellular rhBMP-2 has been associated with many negative side effects including inflammation and ectopic bone formation.

Neither approach for bone growth stimulation is ideal and there is a need for better methods for inducing spinal fusion and other orthopedic reconstructions that avoids iliac crest donor site morbidity or circumvents side effects from recombinant human BMP-2 administrations.

### SUMMARY OF THE INVENTION

Cell compositions for tissue engineering are provided which contain a population of autologous or single donor, minimally passaged fibroblasts, preferably dermal fibroblasts, or a subset of fibroblasts which are pluripotent or have been induced to be pluripotent, in combination with a tissue engineering matrix, scaffold or formulation forming a matrix or scaffold. The tissue engineering matrix or scaffold is preferably porous and biodegradable and has a mean pore diameter or interstitial spacing between 100 μm and 800 μm. The tissue engineering matrix can be a polymer based matrix or scaffold, a hydrogel, a ceramic, or a combination thereof. The population of fibroblasts is preferably at least 90%, 95%, 97%, or 98% pure fibroblasts, wherein the majority of other cell types present in the initial biopsy have been removed, typically by passaging in cell culture. The population is most preferably autologous, obtained by a biopsy, and minimally passaged in cell culture. As used herein, minimally passaged means passaged between two and five times, most preferably three times. In one embodiment, the population of fibroblasts is genetically engineered to secrete a therapeutic protein in an amount effective to induce tissue growth or tissue repair when the cell composition is transplanted into a subject in need thereof. For example, the therapeutic protein can be a bone morphogenic protein when the tissue to be treated is bone tissue. A preferred bone morphogenic protein is BMP-2.

When the cell composition containing the genetically engineered cells is transplanted into a subject, the cells secrete an effective amount of BMP-2 into a host to induce bone growth or bone repair. The cells can secrete the therapeutic protein for days, weeks, or months. It has been discovered that human dermal fibroblasts permanently transduced with lentiviruses to express BMP-2 not only induced more bone formation than recombinant BMP-2, but also induced significantly less acute inflammation 24 hours after surgery. This 24 hours post-surgery time point is an important period of safety concern following cervical spinal fusion using the current clinical standard of practice (recombinant BMP2/infuse), in that the inflammation around the respiratory pathway prevents the patient breathing. The combination of cells secreting BMP-2 with the tissue engineering matrix or scaffold aids in delivering the appropriate amount of BMP-2 to the region of injury to avoid undesirable side effects.

Methods for inducing tissue growth or regeneration include administering the cell compositions to a subject in need an amount effective to induce tissue growth or regeneration. For example, the cell compositions can be used to treat wounds, skin disorders, muscle disorders, bone disorders, neurological disorders, and cardiac disorders.

One embodiment provides a method for inducing spinal fusion of vertebra in a subject. The cell compositions are administered between vertebra to be fused in a subject in an amount effective to induce bone growth. Still another method provides treating a bone disorder or disease in a subject in need thereof by administering to the subject an effective amount of the disclosed cell compositions. The bone disorder or disease can be osteoporosis, osteopenia, osteonecrosis, fracture, non-union fracture, mal-union fracture, delayed union fractures, compression fracture, maxillo-facial fractures, bone reconstruction, cranio-facial bone reconstruction, osteogenesis imperfecta, osteolytic bone cancer, Paget's Disease, endocrinological disorders, hypophsophatemia, hypocalcemia, renal osteodystrophy, osteomalacia, adynamic bone disease, rheumatoid arthritis, hyperparathyroidism, primary hyperparathyroidism, secondary hyperparathyroidism, periodontal disease, Gorham-Stout disease and McCune-Albright syndrome.

Kits containing the cell compositions are also provided. The kits typically include the material forming the cell matrix or scaffold, reagents for resuspending lyophilized or frozen cells, and means for introducing the resuspended cells into the matrix or scaffold.

An object of the present invention to provide cellular compositions for tissue engineering, especially genetically engineered autologous cell compositions.

It is still another object of the invention to provide cellular compositions for tissue engineering for the localized delivery of therapeutic proteins, especially bone morphogenic proteins.

Further aspects and objects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a flow diagram of an exemplary method for inducing in vivo osteogenesis using BMP-2.

FIG. 2 is a graph and Kruskal Wallis comparison results showing differences in inflammation associated with implantation of skin cells transduced BMP-2 by lentivirus and rhBMP-2 treatments.

### DETAILED DESCRIPTION OF THE INVENTION

I. Definitions

As used herein, “minimally-passaged” fibroblasts refers to fibroblasts that have been passaged a smaller number of passages in comparison to prior methods. For example, in generating 3×10⁸ or more cells in a single 10-layer stack, the cells have been passaged no more than three times. In embodiments in which the cells have been further cultured into additional 10-layer stacks, the cells may have undergone additional passages, such as up to a total of 4, 5, or 6 passages in generating 1×10⁹ or more cells.

“Mesenchymal stem cell” or “MSC” refer to multipotent stem cells present in or derived form mesenchymal tissue that can differentiate into a variety of cell types, including: osteoblasts, chondrocytes, and adipocytes.

“Cell” refers to individual cells, cell lines, primary cultures, or cultures derived from such cells unless specifically indicated. “Culture” refers to a composition including isolated cells of the same or a different type. “Cell line” refers to a permanently established cell culture that will proliferate indefinitely given appropriate fresh medium and space, thus making the cell line “immortal.” “Cell strain” refers to a cell culture having a plurality of cells adapted to culture, but with finite division potential. “Cell culture” is a population of cells grown on a medium such as agar.

The terms “primary cells”, “primary cell lines”, and “primary cultures” are used interchangeably herein to refer to cells and cells cultures that have been derived from a subject and allowed to grow in vitro for a limited number of passages, that is, splittings, of the culture. For example primary cultures are cultures that have been passaged 0 times, 1 time, 2 times, 4 times, 5 times, 10 times, or 15 times, but not enough times to go through the crisis stage. Typically, the primary cell lines can be maintained for fewer than 10 passages in vitro. “Primary skin cell culture” refers to a primary cell culture derived from skin cells. A cell can be in vitro or ex vivo. Alternatively, a cell can be in vivo and can be found in a subject. A cell can be a cell from any organism including, but not limited to, animals, preferably humans.

The terms “differentiated somatic cell” or simply “somatic cell” encompass any cell in or of an organism that cannot give rise to all types of cells in an organism. In other words, somatic cells are cells that have differentiated sufficiently that they will not naturally generate cells of all three germ layers of the body, that is, ectoderm, mesoderm and endoderm. For example, somatic cells would include both neurons and neural progenitors, the latter of which are able to naturally give rise to all or some cell types of the central nervous system but cannot give rise to cells of the mesoderm or endoderm lineages.” Examples of somatic cells include those from ectodermal (for example, keratinocytes), mesodermal (for example, fibroblast), endodermal (for example, pancreatic cells), or neural crest lineages (for example, melanocytes). Somatic cells include, for example, dermal fibroblasts, keratinocytes, pancreatic beta cells, neurons, oligodendrocytes, astrocytes, hepatocytes, hepatic stem cells, cardiomyocytes, skeletal muscle cells, smooth muscle cells, hematopoietic cells, osteoclasts, osteoblasts, pericytes, vascular endothelial cells, Schwann cells, and the like. Somatic cells are cells that, in the absence of experimental manipulation, will not proliferate; or if they do, will only be able to give rise to more of their own kind (for example, terminally differentiated cells). Somatic cells can be cells that are differentiated to the point that they are capable of giving rise to cells of a specific lineage (for example, adult non-pluripotent multipotent stem cells, such as mesenchymal stem cells, neural stem cells, cardiac stem cells, hepatic stem cells, and the like). Somatic cells can have a phenotype reflective of their differentiated state (for example, markers, cell morphology, and/or functional characteristics that reflect the differentiated state of the cells).

Isolated,” “isolating,” “purified,” “purifying,” “enriched,” and “enriching,” when used with respect to cells, indicate that the cells at some point in time were separated, enriched, sorted, differentially proliferated, etc., from or with respect to other cells resulting in a higher proportion of the cells compared to the other cells. “Highly purified,” “highly enriched,” and “highly isolated,” when used with respect to cells, indicates that the cells of interest are at least about 70%, about 75%, about 80%, about 85% about 90% or more of the cells, about 95% or more of the cells, and can preferably be about 95% or more of the cells. “Substantially isolated,” “substantially purified,” and “substantially enriched,” when used with respect to cells, indicate that the cells of interest are at least about 70%, about 75%, or about 80% of the cells, more usually at least 85% or 90% of the cells, and sometimes at least 95% or more of the cells, for example, 95%, 96%, and up to 100% of the cells.

“Population,” when used with respect to cells, refers to a group or collection of cells that share one or more characteristics. The term “subpopulation,” when used with respect to cells, refers to a population of cells that are only a portion or subset of a population of cells.

“Passaging” and “passage,” when used with respect to cells, refer to replacing the culture media or transferring cells to new culture media.

“Skin-derived cell” refers to cells isolated from skin tissue and cells cultured, passaged, differentiated, induced, etc., from cells isolated from skin tissue.

“Derived from,” when used with respect to cells, refer to cells isolated from tissue and cells cultured, passaged, differentiated, induced, etc., from cells isolated from tissue.

“Pluripotency” refers to the ability of cells to differentiate into multiplel types of cells in an organism. By “pluripotent stem cells”, it is meant cells that can self-renew and differentiate to produce all types of cells in an organism. By “multipotency” it is meant the ability of cells to differentiate into some types of cells in an organism but not all, typically into cells of a particular tissue or cell lineage.

“Bind,” bound,” “binds to,” and “binding,” when used with respect to cell surface markers, refer to detectable binding of a molecule with a binding affinity and/or specificity for a cell surface marker. A cell having a cell surface marker, the binding of which to a molecule with a binding affinity and/or specificity for a cell surface marker is detectable, can be said to bind to the molecule. By “selectively bind” is meant that the molecule binds preferentially to the target of interest or binds with greater affinity to the target than to other molecules. For example, an antibody can bind to a molecule that includes an epitope for which it is specific and not to unrelated epitopes.

“Express,” “expression,” and “expressing,” when used with respect to gene products, indicate that the gene product of interest is expressed to a detectable level. “Significant expression” refers to expression of the gene product of interest to 10% above the minimum detectable expression. Cells with “high expression” or “high levels” of expression of a given expression product are the 10% of cells in a given sample or population of cells that exhibit the highest expression of the expression product. Cells with “low expression” of a given expression product are the 10% of cells in a given sample or population of cells that exhibit the lowest expression of the expression product (which can be no expression).

A cell “can differentiate into” a specified type of cell if, under conditions that induce differentiation of cells known to differentiate into the specified type of cell, the cell differentiates into the specified type of cell. A cell “does not differentiate into” a specified type of cell if, under conditions that induce differentiation of cells known to differentiate into the specified type of cell, the cell fails to differentiates into the specified type of cell. The ability to differentiate into a specified type of cell (or the lack of such ability) can be limited to one or several differentiation conditions. Thus, for example, a cell could be characterized as capable of differentiating into an osteoblast under the conditions used in the hMSC osteogenic differentiation BulletKit™ (Lonza, Cat. No. PT-3002), even though the cell might not differentiate into osteoblasts under different differentiation conditions. “Conditions that induce differentiation” of cells into a specified type of cell are conditions that cause cells known or established to differentiate to the specified cell type to differentiate into the specified cell type.

“Skin regeneration cell” refers to cells that mediate skin regeneration. “Mediate regeneration” and “participate in repair,” when used with respect to cells, refer to cells that cause or aid in regeneration and/or repair of tissue. For example, cells that mediate skin regeneration can be the source of new skin cells and/or can stimulate other cells to be the source of new skin cells.

“Single donor” refers to cells obtained from one individual, which can be assessed for homogeneity of proteins expressed by the cells, and cell surface markers. “Autologous” refers to single donor cells which are intended for administration back to the host from which the cells were originally obtained.

By “treatment” and “treating” is meant the medical management of one or more symptoms of a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes prophylactic or palliative treatment, that is, treatment designed for the relief of one or more symptoms of the disease, pathological condition, or disorder; preventative treatment, that is, treatment directed to minimizing or partially or completely inhibiting one or more symptoms of the associated disease, pathological condition, or disorder; and supportive treatment, that is, treatment employed to supplement another specific therapy directed toward the improvement of one or more symptoms of the associated disease, pathological condition, or disorder. The effects of treatment can be measured or assessed as described herein and as known in the art as is suitable for the disease, pathological condition, or disorder involved.

“Effective amount” of a cell, device, composition, or compound refers to a nontoxic but sufficient amount of the cell, device, composition, or compound to provide the desired result. The exact amount required may vary from subject to subject, depending on the species, age, and general condition of the subject, the severity of the disease that is being treated, the particular cell, device, composition, or compound used, its mode of administration, and other routine variables. An appropriate effective amount can be determined by one of ordinary skill in the art using only routine experimentation.

“Pharmaceutically acceptable” refers to formulations, devices and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Biocompatible” refers to one or more materials that are neither themselves toxic to the host nor degrade (if the material degrades) at a rate that produces monomeric or oligomeric subunits or other byproducts at toxic concentrations in the host.

“Biodegradable” means that the materials degrades or breaks down into its component subunits by a biochemical process.

“Multi-potent or adult stem cells” are any type of stem cell that is not derived from an embryo or fetus and generally having a limited capacity to generate new cell types (referred to as “multipotency”) and being committed to a particular lineage. Examples of adult stem cells are adipose-derived mesenchymal stem cells and multipotent hematopoietic stem cells. Multipotent hematopoietic stem cells form all of the cells of the blood, such as erythrocytes, macrophages, T and B cells. Cells such as these are referred to as “pluripotent hematopoietic stem cell” for its pluripotency within the hematopoietic lineage.

The term “induced pluripotent stem cell” encompasses pluripotent stem cells, that, like embryonic stem (ES) cells, can be cultured over a long period of time while maintaining the ability to differentiate into all types of cells in an organism, but that, unlike ES cells (which are derived from the inner cell mass of blastocysts), are derived from somatic cells. Generally, pluripotent stem cells are cells that had a narrower, more defined potential and that, in the absence of experimental manipulation, could not give rise to all types of cells in the organism. iPS cells have a hESC-like morphology, growing as flat colonies with large nucleo-cytoplasmic ratios, defined borders and prominent nuclei. In addition, iPS cells can express one or more key pluripotency markers known to those of skill in the art, including but not limited to Alkaline Phosphatase, SSEA3, SSEA4, Sox2, Oct314, Nanog, TRA1S0, TRA181, TDGF 1, Dnmt3b, FoxD3, GDF3, Cyp26a1, TERT, and zfp42. In addition, the iPS cells can be capable of forming teratomas. In addition, iPS cells can be capable of forming or contributing to ectoderm, mesoderm, or endoderm tissues in a living organism.

By “having the potential to become iPS cells” it is meant that somatic cells can be induced to become iPS cells or to redifferentiate so as to establish cells having the morphological characteristics, growth ability and pluripotency of pluripotent cells.

The term “efficiency of reprogramming” is used to refer to the ability of a primary cell culture to give rise to iPS cell colonies when contacted with reprogramming factors. By “enhanced efficiency of reprogramming” it is meant that the cells will demonstrate an enhanced ability to give rise to iPS cells when contacted with reprogramming factors relative to a control.

“Reprogramming factors” refers to one or more factors (that is, a cocktail) of biologically active factors that act on a cell to alter transcription, thereby reprogramming a cell to multipotency or to pluripotency. Reprogramming factors can be provided to cells individually or as a single composition (that is, as a premixed composition) of reprogramming factors. The factors can be provided at the same molar ratio or at different molar ratios. The factors can be provided once or multiple times in the course of culturing the cells. The reprogramming factor can be a transcription factors, including without limitation, Oct3/4; Sox2; Klf4; c-Myc; Nanog; and Lin-28.

The term “bone-related disorder” as used herein refers to any type of bone disease, the treatment of which may benefit from the administration of the cell compositions. For example, the bone disease can be decreased bone formation or excessive bone resorption, by decreased number, viability or function of osteoblasts or osteocytes present in the bone, decreased bone mass in a subject, thinning of bone, compromised bone strength or elasticity, etc. By way of example, but not limitation, bone-related disorders which can benefit from administration of cell compositions may include local or systemic disorders, such as, any type of osteoporosis or osteopenia, e.g., primary, postmenopausal, senile, corticoid-induced, any secondary, mono- or multisite osteonecrosis, any type of fracture, e.g., non-union, mal-union, delayed union fractures or compression, conditions requiring bone fusion (e.g., spinal fusions and rebuilding), maxillo-facial fractures, bone reconstruction, e.g., after traumatic injury or cancer surgery, cranio-facial bone reconstruction, osteogenesis imperfecta, osteolytic bone cancer, Paget's Disease, endocrinological disorders, hypophsophatemia, hypocalcemia, renal osteodystrophy, osteomalacia, adynamic bone disease, rheumatoid arthritis, hyperparathyroidism, primary hyperparathyroidism, secondary hyperparathyroidism, periodontal disease, Gorham-Stout disease and McCune-Albright syndrome.

II. Cellular Compositions for Tissue Engineering

Cellular compositions for tissue engineering are providedwhich include cells in combination with a tissue engineering matrix, scaffold or support.

A. Sources of Cells

1. Autologous Dermal Fibroblasts

The cells in the compositions display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells express proteins characteristic of normal fibroblasts including the fibroblast-specific marker, CD90 (Thy-1), a 35 kDa cell-surface glycoprotein, and the extracellular matrix protein, collagen. CD146 can also be used as a biomarker. The autologous fibroblasts are grown from a biopsy of each individual's own skin using standard tissue culture procedures. Skin tissue (dermis and epidermis layers) is typically biopsied from a patient's post-auricular area. Any tissue containing dermal fibroblasts can be biopsied to produce the suspension of dermal fibroblasts. A preferred method for producing dermal fibroblast suspensions is described in U.S. Pat. No. 8,529,883 to Maslowski.

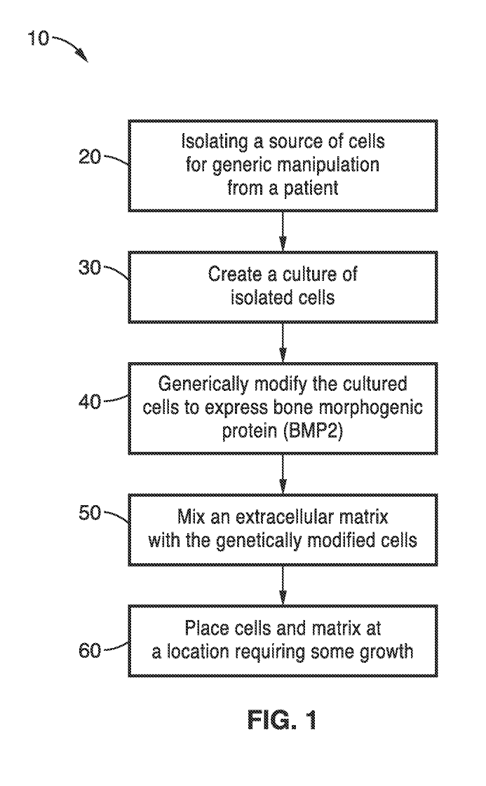

FIG. 1 is a flow diagram of an exemplary method 10 for inducing in vivo osteogenesis using genetically modified cells from the patient. At block 20 of FIG. 1, cells from the patient are acquired, grown and isolated for genetic manipulation. Fibroblast cells obtained from a skin biopsy are particularly preferred because of the ease of acquisition of the sample and the ease of genetic manipulation. Although skin cells are preferred, it will be understood that other cell types from other locations such as stem cells and induced pluripotent stem cells can be used. The skin-derived, patient-specific cells along with the various biomarker-identified subpopulations that can be derived from them can be collected by a variety of methods, including whole biopsy, biopsy fragments and from collagenase treated biopsies.

Other cells that can be used include, but are not limited to, parenchymal cells such as hepatocytes, pancreatic islet cells, chondrocytes, osteoblasts, exocrine cells, cells of intestinal origin, bile duct cells, parathyroid cells, thyroid cells, cells of the adrenal-hypothalamic-pituitary axis, heart muscle cells, kidney epithelial cells, kidney tubular cells, kidney basement membrane cells, nerve cells, blood vessel cells, cells forming bone and cartilage, and smooth and skeletal muscle cells.

2. Precursor Cells

The fibroblasts can also be used to create other cell types for tissue repair or regeneration. Derivation of embryonic stem (ES) cells genetically identical to a patient by somatic cell nuclear transfer (SCNT) holds the potential to cure or alleviate the symptoms of many degenerative diseases while circumventing concerns regarding rejection by the host immune system. Byrne, et al. Nature 2007 Nov. 22;450(7169):497-502, used a modified SCNT approach to produce rhesus macaque blastocysts from adult skin fibroblasts, and successfully isolated two ES cell lines from these embryos. DNA analysis confirmed that nuclear DNA was identical to donor somatic cells and that mitochondrial DNA originated from oocytes. Both cell lines exhibited normal ES cell morphology, expressed key stem-cell markers, were transcriptionally similar to control ES cells and differentiated into multiple cell types in vitro and in vivo. See also Sparman, et al. Stem Cells 2009;27(6):1255-64.

The fibroblasts can be de-differentiated into pluripotent cells: cell fusion (Cowan et al. Science. 2005 Aug. 26;309(5739):1369-73), direct reprogramming (Takahashi, et al., Cell. 2007 30;131(5):861-72), and somatic cell nuclear transfer (Byrne, et al. 2007). Takahashi, et al. demonstrated the generation of iPS cells from adult human dermal fibroblasts with the same four factors: Oct3/4, Sox2, Klf4, and c-Myc. Human iPS cells were similar to human embryonic stem (ES) cells in morphology, proliferation, surface antigens, gene expression, epigenetic status of pluripotent cell-specific genes, and telomerase activity. Furthermore, these cells could differentiate into cell types of the three germ layers in vitro and in teratomas. These findings demonstrate that iPS cells can be generated from adult human fibroblasts.

3. Skin or Fibroblast Cell Derived Stem Cells

Other cells that can be used in the compositions include skin cell derived stem cells. For example skin-cell derived mesenchymal stem cells can be used. These cells are characterized by expression of the cell surface biomarkers CD146 and CD271 and can be more easily obtained, stored, cultured, expanded, and/or differentiated than other multipotent cells due, it is believed, to the convenience of their isolation and their relative purity.

4. SERA Cells

Skin-cell derived SSEA3-expressing regeneration-associated (SERA) cells characterized by expression of the cell surface biomarkers SSEA3 and CD105 (clone 35) and also be used in the compositions. SERA cells can be derived from the dermis of human skin (or other mammalian skin) by selecting, sorting, or enriching for cells expressing SSEA3 and bound by anti-human CD105 antibody clone 35.

B. Preparation of Cells

A biopsy from a subject is obtained and washed several times in a wash media containing IMDM medium with antibiotic agents, such as gentamicin (antibacterial) at a concentration of between 20-40 mg/mL, preferably about 30 mg/mL, and amphotericin B (antifungal) at a concentration of between 10-20 μg/mL, preferably about 15 μg/mL. The biopsy specimen is then digested using a solution of a dissociative or digestive enzyme, and vortexed in an orbital shaker. In one embodiment, the enzyme is trypsin. In another embodiment, the enzyme is a collagenase enzyme, preferably liberase. Growth medium is than added to neutralize the enzyme (when such neutralization is necessary), and the cells are pelleted in a centrifuge. Preferably, growth medium includes IMDM (containing HEPES and L-glutamine and the aforementioned antibiotics) and 10% fetal bovine serum (FBS), although variations in growth medium will be appreciated by those of skill in the art.

Once the harvesting of suitable cells from the patient is accomplished at block 20 of FIG. 1, the isolated cells are cultured at block 30 of FIG. 1 using conventional culturing methods. The optimum method of cell culturing may depend on the type of cells that are isolated from the patient and their susceptibility to genetic manipulation. The approximate number of cultured cells available for genetic modification preferably ranges from approximately 5×10⁶ to approximately 5×10⁷ as a minimum.

In an alternative embodiment, subpopulations of the pool of harvested cells that have identifiable secondary genetic or morphological characteristics that can be used as biomarkers can be segregated from the pool of harvested cells for use. Such subpopulations may have expression patterns or structures that may enhance their effectiveness or act as identifying features.

The cells are resuspended in growth medium, and pipetted into a large tissue culture flask along with sufficient growth medium to keep all cells submerged. A “large” tissue culture flask is one which is at least the size of a T-125 flask, including T-125, T-150, T-175, T-225, T-500, and multilayer culture stacks. The flask is then incubated between 35-39° C. with about 4-6% CO₂. Supplementation of the flask with additional pre-warmed growth medium may be performed at intervals as needed, usually about every 3-5 days. “Pre-warmed” medium is medium which has been warmed after removal from refrigeration, though such medium need not be warmed to physiological temperatures. When supplementing with fresh media, it may be desirable to remove about half of the existing media before adding in the fresh media, which may then be stored as conditioned medium for use elsewhere.

When the cells have reached about 40-100% confluence in the flask, they are passaged into a larger tissue culture flask, such as a T-500 flask or multilayer culture stack. This is accomplished by first removing the growth medium, which now comprises a variety of factors secreted from the growing culture (i.e., it is conditioned medium), which may be stored for use later. The flask is washed with phosphate buffered saline (PBS), then a solution of trypsin-EDTA is used to detach the fibroblasts from the wall of the tissue culture flask, according to procedures known in the art. The detached fibroblasts are suspended in fresh growth media to inactive the trypsin and transferred to a larger flask, such as a T-500 flask or multilayer stack. Again, fresh growth medium may be added to the flask as needed.

If the first passage of cells was into a multilayer stack, or has otherwise produced sufficient cells for treatment, the cells are harvested as described below. Otherwise the same passaging procedure is used when the larger flask reaches about 95-100% confluence, to transfer the cells to a yet larger flask, such as a multilayer culture stack. Once again, additional growth medium may be added as needed and conditioned medium may be stored for later use.

When the cells in the multilayer culture stack have reached about 95-100% confluence, they are harvested, generally yielding at least 1×10⁸ cells, preferably at least 2.0×10⁸ cells, more preferably at least 3.0×10⁸ cells. The cells may be cryopreserved as detailed below, or may be further cultured in additional multilayer culture stacks to generate larger quantities of cells, where desired. For example, the 3×10⁸ or more cells in a 10-layer stack may be split into four additional 10-layer stacks, and cultured to generate more than 1×10⁹ cells.

The cells may then be shipped directly to the point of treatment location, either fresh or cryopreserved, or may be cryopreserved, stored, and shipped at a later date. Preferably, the cells will be suspended in 10-20 mL (this value is also variable depending on the cell population at harvest, typically can be 10-20 mL) of freezing medium (as described below), and transferred to freezing vials, 1.2 mL of suspension per vial. Each vial will thus contain about 2.2×10⁷ cells, sufficient for injection or other administration into a patient. Cryopreserved cells may be shipped frozen, or may be thawed, washed, and resuspended in appropriate media prior to shipping.

Numerous methods for successfully freezing cells for later use are known in the art and are included in the present invention. The frozen storage of early rather than late passage fibroblasts is preferred because the number of passages in cell culture of normal human fibroblasts is limited. The method of as described in the above embodiment results in cells which have only been passaged only once or twice.

C. Genetically Modified Cells

The cells can be genetically modified to secrete a protein, cytokine, growth factor, or combination thereof. The protein to be secreted is selected based on the tissue that is to be treated with the cell compositions. For example, the cells can be genetically modified to secrete bone morphogenic proteins when the tissue to be treated is bone. Techniques for genetically engineering cells to express a desired protein are known in the art. See for example Michael R. Green and Joseph Sambrook, Molecular Cloning: A Laboratory Manual (Fourth Edition) (2012) Cold Spring Harbor Laboratory Press.

In one embodiment, the cells are genetically engineered to secrete therapeutically effective amounts of one or more bone morphogenic proteins such as BMP-2 and BMP-7. Bone morphogenetic proteins (BMPs) are multi-functional growth factors that belong to the transforming growth factor beta (TGFbeta) superfamily. The roles of BMPs in embryonic development and cellular functions in postnatal and adult animals have been extensively studied in recent years. The sequences of the BMPs are well known and available.

The level of expression of the BMP-2 genes is believed to be the determining factor in bone formation rather than cell type. Here, skin fibroblasts are preferably genetically modified by a lentiviral transduction approach to express BMP-2. Although a lentiviral approach is preferred, alternative approaches such as adenoviral vector, episomal plasmid or minicircle techniques can be used to create genetically modified cells expressing BMP-2. The production of bone morphogenic protein can be under the control of an inducible promoter so that the protein is only produced when the inducer molecule is provided to the genetically modified cells.

The genetically modified cells produced at the step at block 40 of FIG. 1 can be associated with a tissue engineering matrix or carrier that can provide additional structure to the ultimate location where the cells are placed at block 60. For example, in one embodiment, the modified cells are embedded within a collagen sponge and the sponge with the cells is placed at the proper location in the body of the patient. In another embodiment, the modified cells are suspended in a collagen solution that can be injected in locations where new bone formation is desired.

Finally, at block 60 of FIG. 1, the genetically modified cells are deposited (with or without the optional tissue engineering matrix) to locations that have been designated for new bone formation or other physiological result. For example, in the case of a spinal fusion, modified cells can be placed between the spinal vertebrae. Locally applied BMP-2 expressing cells are only biologically active over a comparatively short period of time and the stimulus is therefore limited in duration.

At block 60 of FIG. 1, the number of cells that are implanted should produce adequate levels of BMP-2 to promote efficient osteoinduction at the location of deposition. For bone growth in the case of spinal fusion, the minimum number of cells expressing BMP-2 that are implanted to produce adequate levels of BMP-2 was approximately 1.5 million cells to 5 million cells. Therefore, it is preferred that approximately 1.5 million cells or more be implanted to stimulate the desired bone growth. Optimum numbers of genetically modified cells that are ultimately available for use and numbers of transduced cells that express BMP-2 or other proteins that are needed to produce results can be determined by simple experimentation.

III. Matrix or Carrier

The cells can be provided in a matrix formulation or scaffold, or material which forms a matrix or scaffold immediately prior to or at the time of implantation or injection, to facilitate cell survival, maintenance, and/or growth. Implants fabricated from polymers may be used in a wide range of orthopedic and vascular applications, tissue engineering, and guided tissue regeneration. There are tissue engineering applications for virtually every tissue, including bone, liver, cartilage, kidney, lung, skin, heart, bladder, pancreas, bone, uroepithelial-smooth muscle structures (especially ureters and urethras), tracheal epithelium, tendon, breast, arteries, veins, heart valves, gastrointestinal tubes, fallopian tubes, bile ducts, esophagus, and bronchi. Generally the matrix is a three dimensional porous matrix that can be seeded with cells. The average mean pore diameter (in the case of a solid porous matrix) or interstitial spacing (in the case of a polymeric or fibrous matrix) is from 100 micrometers to 800 micrometers. Other materials, such as hydrogels, which are of the same permeability to gases and nutrients as the matrices or scaffold having this porosity or interstitial spacing, may also be used.

The delivery of growth factors, proteins, molecules, and cells (both transfected cells and stem cells) for the purpose of osteogenic bone growth in spinal fusion and in other areas of fracture healing, has required the use of a carrier. This carrier is required to hold the material and allow for delivery of the active materials in the desired area of bone formation. Depending on the area of the body, the different carrier properties should allow for the presence of the active material for the desired activity time, allow for appropriate time release of the material, allow for an appropriate environment for bone formation, and in many areas of the body, is required to biomechanically and physically provide an area for proper bone formation. For spinal fusion, this is often within a biomechanics cage as in intervertebral body fusion, or in the area of posterior inter transverse process spinal fusion, it must physically limit the compressive forces of the paraspinal musculature from negatively affecting bone formation. A variety of carriers have been used such as allograft bone, demineralized bone matrices, ceramics, physical biomaterials and various formulations of allograft bone mixed with physical collagen carrier.

A. Scaffolds

Scaffolds are typically formed of polymeric, ceramic, and/or metals. Polymers may be in the form of fibers, sheets, woven or non-woven structures, hydrogels, or combinations thereof. A hydrogel is defined as a substance formed when an organic polymer (natural or synthetic) is cross-linked via covalent, ionic, or hydrogen bonds to create a three-dimensional open-lattice structure which entraps water molecules to form a gel. Examples of materials which can be used to form a hydrogel include proteins such as fibrinogen, collagen, and hyaluronic acid, polysaccharides such as alginate, polyphosphazines, and polyacrylates, which are crosslinked ionically, or block copolymers such as PLURONICS® or TETRONICS®, polyethylene oxide-polypropylene glycol block copolymers which are crosslinked by temperature or pH, respectively. In general, these polymers are at least partially soluble in aqueous solutions, such as water, buffered salt solutions, or aqueous alcohol solutions that have charged side groups, or a monovalent ionic salt thereof. Examples of polymers with acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

1. Polymeric Scaffolds

Polymeric scaffolds may be formed of natural polymers such as fibrin or collagen or synthetic polymers, typically biodegradable polymers such as polyhydroxy acids like polylactic acid, polyglycolic acid and poly lactic-co-glycolic acid, polyhydroxyalkanoates such as poly(4-hydroxybutyrate), and polyacrylic acids. Other biocompatible, biodegradable materials include, but are not limited to, type 1 collagen, Poly-DL-lactide-caprolactone (PCL), laminin, and gelatin.

A scaffold may be capable of supporting or anchoring embedded cells to an implantation site for a therapeutically effective period of time. This may facilitate blood vessel formation at a given implantation site of a subject. For example, the cells can be provided in a fibrin scaffold as described in U.S. Patent Publication No: 20120039855. In one example, the fibrin scaffold is formed with a fibrinogen component containing fibrinogen having a final concentration of higher than 17.5 mg/ml scaffold which can support a population of cells and/or other derived cells seeded to a concentration of at least 1×10⁶ cells/ml scaffold. The scaffold can also have a biologically active component, For example, the scaffold can include a solution of proteins derived from blood plasma that can also have anti fibrinolytic agents such as tranexamic acid and/or stabilizers such as arginine, lysine, their pharmaceutically acceptable salts, or mixtures thereof. The solution can have additional factors such as, for example, factor VIII, fibronectin, von Willebrand factor (vWF), vitronectin, etc. Examples of this are described in U.S. Pat. No. 6,121,232 and WO 9833533. The solution can also have stabilizers such as tranexamic acid and arginine hydrochloride.

An example of a suitable polymer is polyglactin, which is a 90:10 copolymer of glycolide and lactide, and is manufactured as VICRYL™ braided absorbable suture (Ethicon Co., Somerville, N.J.). Polymer fibers (such as VICRYL™), can be woven or compressed into a felt-like polymer sheet, which can then be cut into any desired shape. Alternatively, the polymer fibers can be compressed together in a mold that casts them into the shape desired for the support structure. In some cases, additional polymer can be added to the polymer fibers as they are molded to revise or impart additional structure to the fiber mesh. For example, a polylactic acid solution can be added to this sheet of polyglycolic fiber mesh, and the combination can be molded together to form a porous support structure. The polylactic acid binds the crosslinks of the polyglycolic acid fibers, thereby coating these individual fibers and fixing the shape of the molded fibers. The polylactic acid also fills in the spaces between the fibers. Thus, porosity can be varied according to the amount of polylactic acid introduced into the support. The pressure required to mold the fiber mesh into a desirable shape can be quite moderate. All that is required is that the fibers are held in place long enough for the binding and coating action of polylactic acid to take effect.

2. Extracellular Matrix

Other natural scaffold materials include extracellular matrix material (ECM). Such extracellular matrix materials are well known to those of skill in the art (see, for example, Halstenberg et al. (2002) Biomacromolecules, 3: 710-723; Mann et al. (2001) Biomaterials, 22: 3045-3051, and the like). Illustrative synthetic ECM materials include, for example, hydrogel ECMs formed from biological materials (for example, hyaluronic and collagen hydrogels, see, for example, HyStem® hydrogels) or ECMs formed from synthetic hydrogel materials (for example, PEG-tetravinylsulfone, see, for example, Lutolf et al. (2003) Proc. Natl. Acad. Sci., USA, 100(9): 5413-5418).

Synthetic skin is typically a matrix of fibers that forms interstices. This can include a therapeutic component such as a cellular component and, optionally, a non-cellular component. The cellular component can include, for example, SERA cells, and/or cells derived from the SERA cells, CD271-MSCs, or both.

Porous matrix materials can also be used. These may be microparticles or microcapsules that have sufficient porosity to allow permeation by the cells, and diffusion of gases and nutrients to maintain the viability of the seeded cells following implantation.

B. Implants and Tissue Engineering Devices

The cells can also be administered adhered to and/or dispersed in devices including, but not limited to, sutures, meniscus repair or regeneration devices, bone plates and bone plating systems, surgical mesh, repair patches, slings, cardiovascular patches, orthopedic pins (including bone filling augmentation material), heart valves and vascular grafts, adhesion barriers, stents, guided tissue repair/regeneration devices, articular cartilage repair devices, nerve guides, tendon repair devices, atrial septal defect repair devices, pericardial patches, bulking and filling agents, vein valves, bone marrow scaffolds, meniscus regeneration devices, ligament and tendon grafts, ocular cell implants, spinal fusion cages, skin substitutes, dural substitutes, bone graft substitutes, bone dowels, wound dressings, and hemostats. The devices can be used for joining or fusing parts of one or more bones, joining tissue to bone, or joining tissue to tissue. The devices can also be used as a framework or scaffold for tissue growth. Such devices are useful, for example, for tissue replacement and regeneration. The devices and implants can be made of synthetic materials, natural materials, or a combination thereof. For example, in the devices can contain segments prepared from natural materials, synthetic materials (including polymers and ceramics), metals, metal alloys, or a combination thereof. In some forms, the device or implant can be made of titanium, typically with porosity and attachment materials for the cells.

For bone and cartilage repair, regeneration, and/or replacement of bone, tendons, and cartilage, the device can include osteoblasts, osteocytes, or both. The devices can also include natural materials. As used herein, “natural material” can be any material derived from a natural source. For example, the natural material can be bone and cartilage, including bone and cartilage harvested from humans or animals. The bone can also be one or more bone products that have been partially or completely demineralized, prepared for transplantation (for example, via removal of immunogenic proteins), and/or processed by other techniques. Additionally, the implants can be prepared from products made from bone, such as chips, putties, and other similar bone products. Human source bone is preferred for human applications.

For tissue engineering, the cells can be provided with or incorporated onto or into a support structure for construction of a new tissue. Support structures can be meshes, solid supports, tubes, porous structures, and/or a hydrogel. The support structures can be biodegradable or non-biodegradable, in whole or in part. The support can be formed of a natural or synthetic polymer, metal such as titanium, bone or hydroxyapatite, or a ceramic. Natural polymers include collagen, hyaluronic acid, polysaccharides, and glycosaminoglycans. Synthetic polymers include polyhydroxyacids such as polylactic acid, polyglycolic acid, and copolymers thereof, polyhydroxyalkanoates such as polyhydroxybutyrate, polyorthoesters, polyanhydrides, polyurethanes, polycarbonates, and polyesters.

1. Solid Supports

The support structure can be a loose woven or non-woven mesh, where the cells are seeded in and onto the mesh. The structure can include solid structural supports. The support can be a tube, for example, a neural tube for regrowth of neural axons. The support can be a stent or valve. The support can be a joint prosthetic such as a knee or hip, or part thereof, that has a porous interface allowing ingrowth of cells and/or seeding of cells into the porous structure.

The support structure can be a permeable structure having pore-like cavities or interstices that shape and support the hydrogel-cell mixture. For example, the support structure can be a porous polymer mesh, a natural or synthetic sponge, or a support structure formed of metal or a material such as bone or hydroxyapatite. The porosity of the support structure should be such that nutrients can diffuse into the structure, thereby effectively reaching the cells inside, and waste products produced by the cells can diffuse out of the structure.

The support structure can be shaped to conform to the space in which new tissue is desired. For example, the support structure can be shaped to conform to the shape of an area of the skin that has been burned or the portion of cartilage or bone that has been lost. Depending on the material from which it is made, the support structure can be shaped by cutting, molding, casting, or any other method that produces a desired shape. The support can be shaped either before or after the support structure is seeded with cells or is filled with a hydrogel-cell mixture, as described below.

Alternatively, or in addition, the support structure can include other types of polymer fibers or polymer structures produced by techniques known in the art. For example, thin polymer films can be obtained by evaporating solvent from a polymer solution. These films can be cast into a desired shaped if the polymer solution is evaporated from a mold having the relief pattern of the desired shape. Polymer gels can also be molded into thin, permeable polymer structures using compression molding techniques known in the art.

Many other types of support structures are also possible. For example, the support structure can be formed from sponges, foams, corals, or biocompatible inorganic structures having internal pores, or mesh sheets of interwoven polymer fibers. These support structures can be prepared using known methods. Bone cements may be used to form these structures at the time of surgery, where the material is applied to form a porous structure, allowed to harden so that the bone surface is strongly adhered to, then filled with the cell suspension.

2. Hydrogels

The cells can be mixed with a hydrogel to form a cell-hydrogel mixture. This cell-hydrogel mixture can be applied directly to a tissue that has been damaged. For example, as described in U.S. Pat. No. 5,944,754, a hydrogel-cell mixture can simply be brushed, dripped, or sprayed onto a desired surface or poured or otherwise made to fill a desired cavity or device. The hydrogel provides a thin matrix or scaffold within which the cells adhere and grow. These methods of administration can be especially well suited when the tissue associated with a patient's disorder has an irregular shape or when the cells are applied at a distant site (for example, when the cells are placed beneath the renal capsule to treat diabetes).

Alternatively, the hydrogel-cell mixture can be introduced into a permeable, biocompatible support structure so that the mixture essentially fills the support structure and, as it solidifies, assumes the support structure's shape. Thus, the support structure can guide the development and shape of the tissue that matures from the implanted cells, or their progeny, that are placed within it. A hydrogel-based method for generating new tissue using isolated cells is described for example in U.S. Pat. No. 6,171,610.

The support structure can be provided to a patient either before or after being filled with the hydrogel-cell mixture. For example, the support structure can be placed within a tissue (for example, a damaged area of the skin, the liver, or the skeletal system) and subsequently filled with the hydrogel-cell composition using a syringe, catheter, or other suitable device. When desirable, the shape of the support structure can be made to conform to the shape of the damaged tissue. In the following subsections, suitable support structures, hydrogels, and delivery methods are described (cells suitable for use are described above).

The hydrogels should be biocompatible, biodegradable, capable of sustaining living cells, and, preferably, capable of solidifying rapidly in vivo (for example, in about five minutes after being delivered to the support structure). Large numbers of cells can be distributed evenly within a hydrogel; a hydrogel can support approximately 5×10⁶ cells/ml. Hydrogels also allow diffusion so that nutrients reach the cells and waste products can be carried away. A variety of different hydrogels can be used with the disclosed cells and compositions. These include, but are not limited to: (1) temperature dependent hydrogels that solidify or set at body temperature (e.g., PLURONICS™); (2) hydrogels cross-linked by ions (e.g., sodium alginate); (3) hydrogels set by exposure to either visible or ultraviolet light, (e.g., polyethylene glycol polylactic acid copolymers with acrylate end groups); and (4) hydrogels that are set or solidified upon a change in pH (e.g., TETRONICS™). Materials that can be used to form these different hydrogels include, but are not limited to, polysaccharides such as alginate, polyphosphazenes, and polyacrylates, which are cross-linked ionically, block copolymers such as PLURONICS™ (also known as POLOXAMERS™), which are poly(oxyethylene)-poly(oxypropylene) block polymers solidified by changes in temperature, TETRONICS™ (also known as POLOXAMINES™), which are poly(oxyethylene)-poly(oxypropylene) block polymers of ethylene diamine solidified by changes in pH.

For purposes of preventing the passage of antibodies into the hydrogel, but allowing the entry of nutrients, a useful polymer size in the hydrogel is in the range of between 10 and 18.5 kDa. Smaller polymers result in gels of higher density with smaller pores.

a. Ionic Hydrogels

In general, polymers that form ionic hydrogels are at least partially soluble in aqueous solutions (e.g., water, aqueous alcohol solutions that have charged side groups, or monovalent ionic salts thereof). There are many examples of polymers with acidic side groups that can be reacted with cations (e.g., poly(phosphazenes), poly(acrylic acids), and poly(methacrylic acids)). Examples of acidic groups include carboxylic acid groups, sulfonic acid groups, and halogenated (preferably fluorinated) alcohol groups. Examples of polymers with basic side groups that can react with anions are poly(vinyl amines), poly(vinyl pyridine), and poly(vinyl imidazole).

Polyphosphazenes are polymers with backbones consisting of nitrogen and phosphorous atoms separated by alternating single and double bonds. Each phosphorous atom is covalently bonded to two side chains. Useful polyphosphazenes can have a majority of side chains that are acidic and capable of forming salt bridges with di- or trivalent cations. Examples of acidic side chains are carboxylic acid groups and sulfonic acid groups. Bioerodible polyphosphazenes can have at least two different types of side chains: acidic side chains capable of forming salt bridges with multivalent cations, and side chains that hydrolyze in vivo (e.g., imidazole groups, amino acid esters, glycerol, and glucosyl). Bioerodible or biodegradable polymers (i.e., polymers that dissolve or degrade within a period that is acceptable in the desired application (usually in vivo therapy)), will degrade in less than about five years and most preferably in less than about one year, once exposed to a physiological solution of pH 6-8 having a temperature of between about 25° C. and 38° C. Hydrolysis of the side chain results in erosion of the polymer. Examples of hydrolyzing side chains are unsubstituted and substituted imidizoles and amino acid esters in which the side chain is bonded to the phosphorous atom through an amino linkage.

Methods for synthesis and the analysis of various types of polyphosphazenes are described in U.S. Pat. Nos. 4,440,921, 4,495,174, and 4,880,622. Methods for the synthesis of the other polymers described above are known to those of skill in the art. See, for example Concise Encyclopedia of Polymer Science and Engineering, J. I. Kroschwitz, Ed., John Wiley and Sons, New York, N.Y., 1990. Many polymers, such as poly(acrylic acid), alginates, and PLURONICS™ are commercially available.

Water soluble polymers with charged side groups can be cross-linked by reacting the polymer with an aqueous solution containing multivalent ions of the opposite charge, either multivalent cations if the polymer has acidic side groups, or multivalent anions if the polymer has basic side groups. Cations useful for cross-linking the polymers with acidic side groups to form a hydrogel include divalent and trivalent cations such as copper, calcium, aluminum, magnesium, and strontium. Aqueous solutions of the salts of these cations can be added to the polymers to form soft, highly swollen hydrogels.

Anions for cross-linking the polymers to form a hydrogel include divalent and trivalent anions such as low molecular weight dicarboxylate ions, terepthalate ions, sulfate ions, and carbonate ions. Aqueous solutions of the salts of these anions can be added to the polymers to form soft, highly swollen hydrogels, as described with respect to cations.

Ionic polysaccharides, such as alginates or chitosan, can also be used to suspend living cells, including the cells described herein and their progeny. These hydrogels can be produced by cross-linking the anionic salt of alginic acid, a carbohydrate polymer isolated from seaweed, with ions, such as calcium cations. The strength of the hydrogel generally increases with either increasing concentrations of calcium ions or alginate. U.S. Pat. No. 4,352,883 describes the ionic cross-linking of alginate with divalent cations, in water, at room temperature, to form a hydrogel matrix.

The cells can be mixed with an alginate solution, for example, which can be delivered to an already implanted support structure, and which can then solidify in a short time due to the presence of physiological concentrations of calcium ions in vivo. Alternatively, the solution can be delivered to the support structure prior to implantation and solidified in an external solution containing calcium ions.

b. Temperature-Dependent Hydrogels

Temperature-dependent, or thermosensitive, hydrogels can also be used with the disclosed cells. These hydrogels have so-called “reverse gelation” properties, that is, they are liquids at or below room temperature, and gel when warmed to higher temperatures (e.g., body temperature). Thus, these hydrogels can be easily applied at or below room temperature as a liquid and automatically form a semi-solid gel when warmed to body temperature. As a result, these gels are especially useful when the support structure is first implanted into a patient, and then filled with the hydrogel-cell composition. Examples of such temperature-dependent hydrogels are PLURONICS™ (BASF-Wyandotte), such as polyoxyethylene-polyoxypropylene F-108, F-68, and F-127, poly(N-isopropylacrylamide), and N-isopropylacrylamide copolymers.

These copolymers can be manipulated by standard techniques to affect their physical properties such as porosity, rate of degradation, transition temperature, and degree of rigidity. For example, the addition of low molecular weight saccharides in the presence and absence of salts affects the lower critical solution temperature (LOST) of typical thermosensitive polymers. In addition, when these gels are prepared at concentrations ranging between 5 and 25% (WN) by dispersion at 4° C., the viscosity and the gel-sol transition temperature are affected, the gel-sol transition temperature being inversely related to the concentration. These gels have diffusion characteristics capable of allowing cells to survive and be nourished. U.S. Pat. No. 4,188,373 describes using PLURONIC™ polyols in aqueous compositions to provide thermal gelling aqueous systems. U.S. Pat. Nos. 4,474,751 and 4,478,822 describe drug delivery systems that utilize thermosetting polyoxyalkylene gels. With these systems, both the gel transition temperature and/or the rigidity of the gel can be modified by adjustment of the pH and/or the ionic strength, as well as by the concentration of the polymer.

c. pH-Dependent Hydrogels

Other hydrogels suitable for use with the disclosed cells are pH-dependent. These hydrogels are liquids at, below, or above specific pH values, and gel when exposed to specific pHs, for example, 7.35 to 7.45, the normal pH range of extracellular fluids within the human body. Thus, these hydrogels can be easily delivered to an implanted support structure as a liquid and automatically form a semi-solid gel when exposed to body pH. Examples of such pH-dependent hydrogels are TETRONICS™ (BASF-Wyandotte) polyoxyethylene-polyoxypropylene polymers of ethylene diamine, poly(diethyl aminoethyl methacrylate-g-ethylene glycol), and poly(2-hydroxymethyl methacrylate). These copolymers can be manipulated by standard techniques to affect their physical properties.

d. Light Solidified Hydrogels

Other hydrogels that can be used with the disclosed cells are solidified by either visible or ultraviolet light. These hydrogels are made of macromers including a water soluble region, a biodegradable region, and at least two polymerizable regions as described for example in U.S. Pat. No. 5,410,016). The hydrogel can begin with a biodegradable, polymerizable macromer including a core, an extension on each end of the core, and an end cap on each extension. The core can be a hydrophilic polymer, the extensions can be biodegradable polymers, and the end caps can be oligomers capable of cross-linking the macromers upon exposure to visible or ultraviolet light, for example, long wavelength ultraviolet light. Examples of such light solidified hydrogels include polyethylene oxide block copolymers, polyethylene glycol polylactic acid copolymers with acrylate end groups, and 10K polyethylene glycol-glycolide copolymer capped by an acrylate at both ends. As with the PLURONIC™ hydrogels, the copolymers of these hydrogels can be manipulated by standard techniques to modify their physical properties such as rate of degradation, differences in crystallinity, and degree of rigidity.

e. Preparation of Hydrogel-Cell Mixtures

Once a hydrogel of choice is prepared, the cells described herein are suspended in the hydrogel solution. The concentration of the cells suspended in the hydrogel solution can mimic that of the tissue to be generated. For example, the concentration of cells can range between 10 and 100 million cells/ml (e.g., between 20 and 50 million cells/ml or between 50 and 80 million cells/ml). The optimal concentration of cells to be delivered into the support structure can be determined on a case by case basis, and can vary depending on cell type and the region of the patient's body into which the support structure is implanted or onto which it is applied.

f. Administering Hydrogel-Cell Mixtures

The liquid hydrogel-cell mixture can be delivered to a shaped support structure, either before or after the support structure is implanted in or applied to a patient. The specific method of delivery will depend on whether the support structure is sufficiently “sponge-like” for the given viscosity of the hydrogel-cell composition, that is, whether the support structure easily retains the liquid hydrogel-cell mixture before it solidifies. Sponge-like support structures can be immersed within, and saturated with, the liquid hydrogel-cell mixture, and subsequently removed from the mixture. The hydrogel is then allowed to solidify within the support structure. The hydrogel-cell-containing support structure is then implanted in or otherwise administered to the patient. The support structure can also be applied to the patient before the hydrogel completely solidifies. Alternatively, a sponge-like support structure can be injected with the liquid hydrogel-cell mixture, either before or after the support structure is implanted in or otherwise administered to the patient. The hydrogel-cell mixture is then allowed to solidify.

Support structures that do not easily retain the liquid composition require somewhat different methods. In those cases, for example, the support structure is immersed within and saturated with the liquid hydrogel-cell mixture, which is then allowed to partially solidify. Once the cell-containing hydrogel has solidified to the point where the support structure can retain the hydrogel, the support structure is removed from the partially solidified hydrogel, and, if necessary, partially solidified hydrogel that remains attached to the outside of the support structure is removed (e.g., scraped off the structure).

Alternatively, the liquid hydrogel-cell mixture can be delivered into a mold containing the support structure. For example, the liquid hydrogel-cell mixture can be injected into an otherwise fluid-tight mold that contains the support structure and matches its outer shape and size. The hydrogel is then solidified within the mold, for example, by heating, cooling, light-exposure, or pH adjustment, after which, the hydrogel-cell-containing support structure can be removed from the mold in a form that is ready for administration to a patient.

The support structure can also be implanted in or otherwise administered to the patient (e.g., placed over the site of a burn or other wound, placed beneath the renal capsule, or within a region of the body damaged by ischemia), and the liquid hydrogel-cell mixture can then be delivered to the support structure. The hydrogel-cell mixture can be delivered to the support using any simple device, such as a syringe or catheter, or merely by pouring or brushing a liquid gel onto a support structure (e.g., a sheet-like structure).

To apply or implant the support structure, the implantation site within the patient can be prepared (e.g., in the event the support structure is applied to the skin, the area can be prepared by debridement), and the support structure can be implanted or otherwise applied directly at that site. If necessary, during implantation, the site can be cleared of bodily fluids such as blood (e.g., with a burst of air or suction).

3. Ceramics

Ceramic devices are often used in tissue engineering of bone. The requirements for a scaffold in bone regeneration are: (1) biocompatibility, (2) osteoconductivity, (3) interconnected porous structure, (4) appropriate mechanical strength, and (5) biodegradability. An exemplary scaffold is made of interconnected porous hydroxyapatite (IP-CHA) made by adopting the “form-gel” technique (Yoshikowa, H. et al., J Artif Organs, 8(3):131-6 (2005). IP-CHA has a three-dimensional structure with spherical pores of uniform size that are interconnected by window-like holes; the material also demonstrated adequate compression strength. In animal experiments, IP-CHA showed superior osteoconduction, with the majority of pores filled with newly formed bone. The interconnected porous structure facilitates bone tissue engineering by allowing the introduction of bone cells, osteotropic agents, or vasculature into the pores.

Calcium phosphate ceramics (CPCs) can also be used. CPCs have been widely used as biomaterials for the regeneration of bone tissue because of their ability to induce osteoblastic differentiation in progenitor cells. Specific materials that can be used include, but are not limited to hydroxyapatite, calcium phosphate, calcium carbonate, calcium sulfate, tricalcium phosphate (TCP), CaCO₃ (argonite), CaSO₄-2H₂0 (plaster of Paris), and Ca₃(PO₄)₂ (beta-whitlockite, a form of tricalcium phosphate, TCP), (Ca₁₀(PO₄)₆(OH)₂), and tetracalcium phosphate.

Many fabrication techniques are available to produce ceramic scaffolds with varying architectural features. These include gas foaming, soluble or volatile poragen processing, phase-mixing, free form fabrication such as strereolithography, and template coating and casting. Highly porous micro-crystalline CaP scaffolds can be prepared by applying the CaP slurry with a compression/release process and thereby forming a uniform surface coating on the template. Following a heat-sintering schedule, the templates are volatilized leaving the sintered ceramic scaffold with controllable crystalline structure.

Once the ceramic support has been formed, the cells can be seeded into the support. The cell composition can be directly implanted in to a subject or the cell composition can be maintained in cell culture until a suitable amount of cell attachment to the support occurs.

C. Additional Therapeutic Agents

Additional factors, such as growth factors, other factors that induce differentiation or dedifferentiation, secretion products, immunomodulators, anti-inflammatory agents, regression factors, biologically active compounds that promote innervation or enhance the lymphatic network, and drugs, can be incorporated into the tissue engineering matrix or scaffold.

IV. Diseases and Disorders to be Treated

The cellular compositions can be used to treat a variety of tissue or organ diseases or disorders. The disease to be treated will depend on the type of cell used and the therapeutic agent secreted from the cells. Cardiac diseases and disorders can be treated to regenerate cardiac tissue or to replace damaged cardiac tissue. Diseases of the skin can be treated using the cellular compositions including burns or other disorder requiring skin replacement.

Disorders of the bone and bone injuries can be treated with the cell compositions expressing a factor such as bone morphogenic protein, especially BMP-2. The bone disease can be decreased bone formation or excessive bone resorption, by decreased number, viability or function of osteoblasts or osteocytes present in the bone, decreased bone mass in a subject, thinning of bone, compromised bone strength or elasticity, etc. By way of example, but not limitation, bone-related disorders which can benefit from administration of cell compositions may include local or systemic disorders, such as, any type of osteoporosis or osteopenia, e.g., primary, postmenopausal, senile, corticoid-induced, any secondary, mono- or multisite osteonecrosis, any type of fracture, e.g., non-union, mal-union, delayed union fractures or compression, conditions requiring bone fusion (e.g., spinal fusions and rebuilding), maxillo-facial fractures, bone reconstruction, e.g., after traumatic injury or cancer surgery, cranio-facial bone reconstruction, osteogenesis imperfecta, osteolytic bone cancer, Paget's Disease, endocrinological disorders, hypophsophatemia, hypocalcemia, renal osteodystrophy, osteomalacia, adynamic bone disease, rheumatoid arthritis, hyperparathyroidism, primary hyperparathyroidism, secondary hyperparathyroidism, periodontal disease, Gorham-Stout disease and McCune-Albright syndrome.

V. Kits

The cellular compositions can be assembled into a kit. The kit includes a container that holds the components of the kit. Components of the kit can include frozen dermal fibroblasts, for example fibroblasts that are at least 98% pure dermal fibroblasts. The kit can also contain the ingredients for forming a tissue engineering matrix to be used with the dermal fibroblasts. In one embodiment, the tissue engineering matrix is pre-formed in the kit. The kit can also contain materials for transfecting the dermal fibroblasts to secrete a therapeutic protein of interest, for example BMP-2. In some embodiments, the dermal fibroblasts in the kit are genetically engineered to express the therapeutic protein of interest.

VI. Examples and Results

Recombinant Fibroblast Expressing BMP-2

An in vitro culture of primary human skin cells was prepared and the cells genetically modified to express BMP-2. Biopsy fragments that were obtained from a 4 mm adult skin punch biopsy were used to derive a human fibroblast (HUF1) primary cell line. The sample of human skin cells can be derived by a variety of different methods, including whole biopsy, biopsy fragments and from collagenase treated biopsies. No difference in the ability of human skin cells to express BMP-2 to induce spinal fusion is anticipated as a result of the methodology that is used to derive the human skin cells or whether large or small amounts of tissue are used.

All biopsy-derived human skin cells were cultured in regular cell culture media that consisted of Dulbecco's modified Eagle medium nutrient mixture F-12 (DMEM/F12) supplemented with 10% fetal bovine serum (FBS; Invitrogen), 1% MEM nonessential amino acids, 2 mM GlutaMAX, and 100 IU/mL penicillin-streptomycin (Invitrogen). The culture media was changed every 2 days. The cells were allowed to expand to >90% confluency before passaging with 0.05% trypsin-EDTA (Invitrogen) and replating at 1250-1500 cell/cm².

The cultured cells were evaluated for CD146 expression, stained and sorted with a fluorescence activated cell sorting-based purification. Human skins cells contain a population of CD146 expressing cells and CD146 non-expressing cells.

Approximately 4.5×10⁷ cells were trypsinized and washed twice with ice-cold phosphate-buffered saline (PBS) +2% goat serum (PBS-G). The cells were then passed through a 40 μm filter to remove any clumps. After the washes, the cells were resuspended in 0.1 mL (per 4-5×10⁶ cells) of ice-cold PBS-G containing 1:100 CD146:FITC antibody (Abd Serotec, MCA2141 F) and incubated for 30 minutes in the dark at 4° C. with gentle rocking. The incubated cells were washed three times with ice-cold PBS-G, resuspended in 1 mL of ice-cold PBS-G, passed through a 40 μm filter, and immediately analyzed and sorted on a FACSAria cell sorter (BD Biosciences). Data were analyzed and DAPI-stained dead-cell exclusion and doublet-exclusion gating were performed. Viable single-cell subpopulations were sorted using BD FACSDiva Software (BD Biosciences).

FACS-purified CD146-expressing and CD146-non-expressing human skin cells were recovered independently under standard cell culture conditions and allowed to expand to >90% confluency before the commencement of the hMSC-differentiation protocol (Lonza, PT-3002).

Lentivirus-based vectors encoding CMV driven BMP2-Ires-GFP and MCS-Ires-GFP (control virus) were generated by transient cotransfection of 293T cells with a three-plasmid combination. Viral titer was determined by assessing viral p24 antigen concentration by ELISA (The Alliance® HIV-I p24 ELISA Kit, Perkin Elmer).

At day 20 of Osteogenic-differentiation, 1×10⁷ of CD146-expressing and CD146-non-expressing human skin cells were infected in the presence of 5 μg/mL of Polybrene (Millipore) with a High-MOI (20-30 MOD and a Low-MOI (2-3 MOI expected) virus vector dose. After overnight incubation, the adherent cell-monolayers were scraped and dissociated with 0.05% trypsin-EDTA. The cells were then passed through a 40 μm filter and concentrated into 5×10⁶ aliquots with 100 μL of osteogenic media and then applied to the collagen carrier for surgery.

At 21 days of osteogenesis differentiation, the level of in vitro mineral deposit was assayed using Alizarin Red S [40 mM] pH 4.1-4.5 (Sigma Cat# A5533). Alizarin Red Staining and the quantitative analysis of Alizarin Red Staining were executed according the manufacturer recommendations (Millipore, ECM815). Light microscopy-based imaging was performed with an AxioCam HR Color Camera using AxioVision Digital Image Processing Software (Axio Observer Inverted Microscope, Carl Zeiss). Alizarin Red colorimetric determinations were performed at OD405 pH 4.1-4.5 in 96-well format (Costar, 07-200-568) using the Tecan Infinite® 200 multimode microplate reader provided with Tecan-i-Control Plate reader Analysis Software. (Tecan)/Alizarin Red S data was corroborated with Osteocalcin secretion in the osteogenic cultures.

The cells were fixed in 4% paraformaldehyde/PBS for 20 minutes, washed once with PBS supplemented with 100 mM glycine for 10 minutes, and then washed twice with PBS for 5 minutes each. Blocking was performed with 4% goat serum in Casein-PBS for 1 hour at room temperature. Subsequently, 1:50 Osteocalcin antibody (Santa Cruz, sc-74495) was added to 4% goat serum in casein-PBS and incubated overnight at 4° C. with slow nutation. The next day, the cells were washed thrice with PBS for 5 minutes before a fluorescent-conjugated secondary Alexa 594-conjugated goat anti-mouse IgG (Invitrogen, A11005) was added at 1:500 to 4% goat serum in casein-PBS and incubated for 1 hour at room temperature, protected from light. The cells were rinsed thrice with PBS, and DAPI was used to label the nuclei. A final PBS rinse of the cells for 10 minutes at room temperature was performed. Visualization was performed with an AxioCam MRMonocolor Camera using Axio-Vision Digital Image Processing Software (Axio Observer Inverted Microscope, Carl Zeiss).

Although lentiviruses with CMV promoters were used, no difference in the ability of BMP-2 transgenically modified cells to induce spinal fusion is anticipated as a result of the vector (adenovirus, episomal plasmid etc) and/or promoter (hEF1alpha, UbC etc) that are used as long as the actual amount of BMP-2 being produced by the modified cells is sufficient to produce results.

In addition, it was observed that both the CD146 positive and CD146 negative subpopulations of human skin cells demonstrated the ability to induce spinal fusion following transduction with BMP-2 lentivirus. Therefore, human skin cells can be used regardless of CD146 expression status to induce spinal fusion when transduced by BMP-2.

However, it was also observed that only the high MOI (20-30) group resulted in successful spinal fusion suggesting that a minimum number of cells expressing BMP-2 are necessary to successfully produce sufficient bone growth for fusion. Therefore, transduction efficiency and the number of inserted cells should be optimized to ensure that the BMP-2 is sufficiently high for spinal fusion to occur.

BMP-2 Secreting Skin Cells Induce a Robust Formation of New Bone

To further demonstrate the invention, twenty-four female, athymic rats, twelve weeks of age, were randomly separated into fourteen different treatment groups for in vivo studies. Selected groups were treated with either CD146-positive cells, CD146-negative cells or Recombinant human BMP-2 (rhBMP-2). The cells were incubated with either Lenti-GFP at 20 MOI, Lenti-GFP at 2 MOI, Lenti BMP-GFP at 20 MOI, Lenti BMP-GFP at 2 MOI or without a vector. In each group treated with cells, 5×106 cells that were suspended in 100 μl of αMEM were transplanted into the rat spine and evaluated. Recombinant human BMP-2 (rhBMP-2) (Medtronic, Minneapolis, Minn.) was diluted in phosphate buffered saline solution at a concentration of 0.05 μg/μl and was added to either the cell suspension or in 100 μl αMEM just before transplantation.

A type-I collagen sponge (HELISTAT; Integra LifeSciences, Plainsboro, N.J.) measuring 8×12×2 mm was used as a carrier for the procedures performed with all of the groups. For implantation of a carrier with cells or rhBMP-2, each rat was anesthetized with a continuous isoflurane inhalational anesthetic chamber and monitored for cardiac or respiratory difficulties by an assistant throughout the procedure. After a posterior midline incision was made over the lumbar spine, two separate fascial incisions were made 4 mm from the midline around the spinous processes. The transverse processes of L4 and L5 were then exposed with use of a muscle-splitting approach carried out with blunt dissection down to the periosteum. A high-speed burr was then used to decorticate the exposed transverse processes. Graft materials were saturated with a type-I collagen sponge for approximately five minutes and then were implanted between the transverse processes bilaterally in the paraspinal muscles. The fascial and skin incisions were closed with use of a 2-0 Vicryl (polyglactin) absorbable running suture. The rats were housed in separate cages and allowed to eat, drink, and bear weight without limitations.

Radiographic analysis of the implantation sites was conducted at two, four, six, and eight weeks after treatment. The animals were anesthetized with inhalational isoflurane and plain anteroposterior radiographs were taken using a Faxitron cabinet (Field Emission, McMinniville, Oreg.). Fusion between the L4 and L5 transverse processes in each rat was recorded as the percentage of the total area between L4 and L5 that was filled with new bone. Three blinded independent observers scored the bone formation in each rat according to a 6-point scale: 0, no bone formation; 1, bone filling less than 25% of the area; 2, bone filling 25% to 50% of the area; 3, bone filling 50% to 75% of the area; 4, bone filling 75% to 99% of the area; and 5, clear evidence of fusion with bone filling all gaps between L4 and L5.

Manual assessment of fusion was conducted after eight weeks of treatment. The subject rats were sacrificed and the explanted lumbar spines were manually tested for intersegmental motion by three blinded independent observers. Any motion detected on either sides between the facets or between the transverse processes was considered as a failure of fusion, and unilateral fusion was considered as no fusion. The absence of motion (right and left) and bilateral fusion was considered as successful fusion.

After the manual assessment, the harvested spines were fixed in 10% buffered formalin 40% ethanol for 1 week. Then the spines were scanned using high-resolution micro-computed tomography (micro-CT) that used the 9-20-mm resolution technology of mCT40 (Scanco Medical, Basserdorf, Switzerland). The micro-CT data were collected at 55 kVp and 72 mA and reconstructed using a cone-beam algorithm supplied with the Scanco micro-CT scanner. Visualization and data reconstruction were performed using mCT Ray T3.3 and mCT Evaluation Program V5.0 (Scanco Medical), respectively. Using these software packages, the area from the tip of the L4 transverse process to the base of the L5 transverse process on the micro-CT images were measured in the groups with 100% fusion to compare the volume of new bone formation.

After the micro-CT scan, the specimens were decalcified using standard 10% decalcifying solution HCl (Cal-Ex) (Fisher Scientific, Fairlawn, N.J.), washed with running tap water, and then transferred to 70% ethanol for histological analysis. Serial sagittal sections near the transverse processes were cut carefully at the level of the transverse process. The specimens were embedded in wax and sectioned. The sections were stained with hematoxylin and eosin. Three independent observers blindly scored the histological bone formation. Histologic fusion was defined as bony trabeculae bridging from one transverse process to the next. Fusion masses were assessed and the extent of new bone formation was scored using the following scoring criteria: 0, empty cleft; 1, slight bump within the fibrocartilage tissue (filling less than 25% of the gap area); 2, some gaps within the fibrocartilage tissue (filling 25-50% of the gap area); 3, small gaps within the fibrocartilage and bone tissue (filling 75-99% of the gap area); 4, bridged with bone tissue, however, the fusion masses were composed of thin trabecular bone; and 5, completely bridged with abundant mature bone tissue.

Successful Spinal Fusion was Observed in 75% (3 Out of 4) of the Rats After 1 Month

Having demonstrated that BMP-2 secreting skin cells induce a robust formation of new bone in the rodent model and successfully induce spinal fusion, the success of the transduction of skin cells to express BMP-2 and the number of cells needed to induce spinal fusion were quantified. The level of GFP expression in the transduced human skin cell populations was assayed and it was observed that approximately 30% of the cells had significant expression of GFP. Following injection of 5 million of these cells, successful spinal fusion was observed in 75% (3 out of 4) of the rats after 1 month.

Accordingly, rat spinal fusion can be achieved the majority of the time with the implantation of 5 million cells, with only 30% of the cells actually expressing BMP-2-GFP (as assayed by GFP). Therefore, a minimum of approximately 1.5 million actively expressing cells is needed to predictably produce spinal fusion. It is believed that fluorescence-based purification (FACS, MACS, and LEAP) will both purify the number of cells expressing BMP2-GFP and permit higher rates of spinal fusion.

The results also demonstrated that human skin cells obtained from skin punch biopsies, which is quicker, less invasive and less painful than the surgical aspiration of adipose tissue and represents one of the best sources of cells for transgenic manipulation.

Therefore, isolated and purified populations of human skin cells modified to express BMP-2 will permit spinal fusion when placed in minimum numbers at the proper location. A variety of methods of delivery of the BMP-2 expressing modified cells should now be available such as by injection of cells using an injectable matrix. The use high concentrations of purified BMP-2 producing cells to induce spinal fusion using a single large-scale injection of cells in combination with an injectable extra-cellular matrix could eliminate the costs, complications and inconveniences associated with the performance of spinal surgery.

Cellular BMP-2 Induces Less Acute Inflammation than Recombinant BMP-2

Comparisons of the implantation of cells genetically modified to express BMP-2 with recombinant human BMP-2 (rhBMP-2) exposure in rats described previously suggested that cellular BMP-2 induces less acute inflammation than recombinant BMP-2.

To demonstrate that cellular BMP-2 induces less acute inflammation than recombinant BMP-2 and thus should be safer for cervical spinal fusion and other osteogenic applications where inflammation is a safety concern, an assessment of inflammation associated with implantation of skin cells transduced BMP-2 by lenti-virus was performed.

Three groups of rats were provided for the inflammation assessment. The first group was the SCs Group that was implanted with 5×106 skin cells incubated with the lenti-virus vector. The second group was the Recombinant BMP Group that were administered 10 μg of rhBMP2+ACS. The third group was the control group (ACS).

The implantation site and wound of each rat in the study were evaluated for inflammation and the results of each group were compared with each other and to the control. Evaluations took place on days 1, 2, 3 and 7 from the date of surgery.

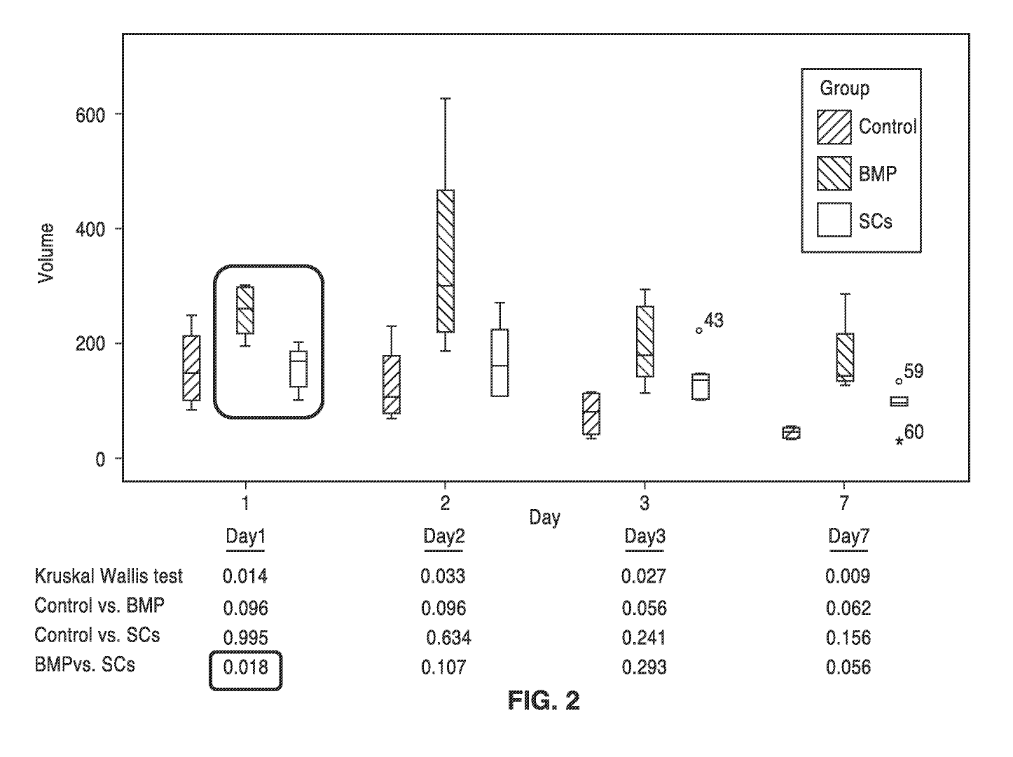

As seen in the results shown in FIG. 2, human skin cells genetically modified to express BMP-2 can successfully induce spinal fusion, while demonstrating significantly less acute inflammation (when compared to rhBMP-2), and as such represents a safer alternative for osteogenic applications where inflammation is a safety concern, such as cervical spinal fusion. Specifically, we noted that the cell-produced BMP2 group induced no additional acute inflammation 24 hours after transplantation compared to the control collagen sponge. While the recombinant BMP2 induced a large amount of inflammation. This is the first discovery, to our knowledge, that cellular produced BMP2 induces no significant additional inflammation (over control baseline) at the critical 24 hour post surgery time point and highlights an important potential application for osteogenic application where excessive acute inflammation is a potential safety concern.

From the discussion above it will be appreciated that the invention can be embodied in various ways, including but not limited to the following:

1. A cell composition comprising a population of single donor, minimally passaged fibroblasts in combination with a tissue engineering matrix or scaffold, or material forming a matrix or scaffold, genetically engineered to express a therapeutic or prophylactic protein.

2. The cell composition of any previous embodiment, wherein the population of fibroblasts are genetically engineered to secrete a therapeutic protein in an amount effective to induce tissue growth or tissue repair when the cell composition is transplanted into a subject in need thereof.

3. The cell composition of any previous embodiment, wherein the therapeutic protein is a bone morphogenic protein.

4. The cell composition of any previous embodiment, wherein the bone morphogenic protein is BMP-2.

5. The cell composition of any previous embodiment, wherein the cell population secretes an effective amount of BMP-2 when transplanted into a host to induce bone growth or bone repair.

6. The cell composition of any previous embodiment, wherein the secretion of BMP-2 by the cell population results in less acute inflammation than administration of BMP-2 directly into the subject.

7. The cell composition of any previous embodiment, wherein the secretion of BMP-2 by the cell population results in less ectopic bone formation than administration of BMP-2 directly to the subject.

8. The cell composition of any previous embodiment, wherein the tissue engineering matrix or scaffold is biodegradable.

9. The cell composition of any previous embodiment, wherein the tissue engineering matrix or scaffold is a hydrogel.

10. The cell composition of any previous embodiment, wherein the tissue engineering matrix or scaffold comprises a ceramic.

11. The cell composition of any previous embodiment, wherein the ceramic is selected from the group consisting of hydroxyapatite, calcium phosphate, calcium carbonate, calcium sulfate, tricalcium phosphate (TCP), CaCO3 (argonite), CaSO4-2H2O (plaster of Paris), and Ca3(PO4)2 (beta-whitlockite, a form of tricalcium phosphate, TCP), (Ca10(PO4)6(OH)2), and tetracalcium phosphate.

12. A method for treating a wound, surgical site, or tissue in need thereof, the method comprising administering the cell composition of any of any previous embodiment to a subject in need thereof in an amount effective to induce tissue growth or tissue regeneration.

13. A method of treating a bone injury, disorder or disease in a subject in need thereof, the method comprising administering to the subject an effective amount of the cell composition of any previous embodiment.

14. The method of any previous embodiment, wherein the bone disorder or disease is selected from the group consisting of osteoporosis, osteopenia, osteonecrosis, fracture, non-union fracture, mal-union fracture, delayed union fractures, compression fracture, maxillo-facial fractures, bone reconstruction, cranio-facial bone reconstruction, osteogenesis imperfecta, osteolytic bone cancer, Paget's Disease, endocrinological disorders, hypophsophatemia, hypocalcemia, renal osteodystrophy, osteomalacia, adynamic bone disease, rheumatoid arthritis, hyperparathyroidism, primary hyperparathyroidism, secondary hyperparathyroidism, periodontal disease, Gorham-Stout disease and McCune-Albright syndrome.

15. A method for inducing spinal fusion of vertebra comprising administering the cell composition of any previous embodiment between vertebra to be fused in a subject in an amount effective to induce bone growth.

16. A kit for use in the method of any previous embodiments, comprising: genetically engineered minimally passaged single donor cells; and a tissue engineering scaffold, matrix, or material forming a matrix.

17. The kit of any previous embodiment wherein the cells are fibroblasts.

18. The kit of any previous embodiment wherein the cells are pluripotent or multipotent fibroblasts.

19. The kit of any previous embodiment wherein the tissue scaffold or matrix is or forms a part of a bone implant or cement.

20. The kit of any previous embodiment wherein the fibroblasts are minimally passaged autologous dermal fibroblasts.

21. A method of stimulating bone formation in the body, the method comprising: genetically modifying autologus cells to express bone formation stimulating proteins; and implanting said genetically modified autologus cells in a location in a body of a patient identified for bone formation to induce bone formation.

22. A method as recited in any previous embodiment, wherein the genetic modification of autological cells comprises culturing a sample of cells from a patient; and incubating cultured cells with an integrating or non-integrating viral vector, said vector containing genes for at least one bone stimulating protein; wherein cells are modified to express bone formation stimulating proteins by transfection of a viral vector.

23. A method as recited in any previous embodiment, wherein the viral vector comprises an integrating lentiviral vector or a non-integrating adenoviral vector.

24. A method as recited in any previous embodiment, wherein the vector is selected from the group of vectors consisting of an adenoviral vector, a miniplasmid vector, a minicircle vector and an episomal plasmid vector.

25. A method as recited in any previous embodiment, wherein the autologus cells are cells selected from the group of cells consisting of human dermal fibroblast cells, adipose tissue cells and stem cells.

26. A method as recited in any previous embodiment, wherein the bone formation stimulating protein is a bone morphogenic protein.

27. A method as recited in any previous embodiment, further comprising: associating an extracellular matrix with the genetically modified cells; and implanting the extracellular matrix and associated cells in the body of a patient.

28. A method as recited in any previous embodiment, wherein the genetic modification of the cultured cells comprises: introducing genes for at least one bone growth stimulating protein into the cultured cells with a vector to produce genetically modified cells; and separating genetically modified cells that express bone growth stimulating proteins from cells that do not.

29. A cell composition comprising a population of single donor, minimally passaged fibroblasts in combination with a tissue engineering matrix or scaffold, or material forming a matrix or scaffold, genetically engineered to express a therapeutic or prophylactic protein.

30. The cell composition of embodiment 29, wherein the population of fibroblasts are genetically engineered to secrete a therapeutic protein in an amount effective to induce tissue growth or tissue repair when the cell composition is transplanted into a subject in need thereof.

31. The cell composition of embodiment 30, wherein the therapeutic protein is a bone morphogenic protein.

32. The cell composition of embodiment 31, wherein the bone morphogenic protein is BMP-2.

33. The cell composition of embodiment 29, wherein the cell population secretes an effective amount of BMP-2 when transplanted into a host to induce bone growth or bone repair.

34. The cell composition of embodiment 33, wherein the secretion of BMP-2 by the cell population results in less acute inflammation than administration of BMP-2 directly into the subject.

35. The cell composition of embodiment 33, wherein the secretion of BMP-2 by the cell population results in less ectopic bone formation than administration of BMP-2 directly to the subject.

36. The cell composition of embodiment 29, wherein the tissue engineering matrix or scaffold is biodegradable.

37. The cell composition of embodiment 29, wherein the tissue engineering matrix or scaffold is a hydrogel.

38. The cell composition of embodiment 29, wherein the tissue engineering matrix or scaffold comprises a ceramic.

39. The cell composition of embodiment 38, wherein the ceramic is selected from the group consisting of hydroxyapatite, calcium phosphate, calcium carbonate, calcium sulfate, tricalcium phosphate (TCP), CaCO₃ (argonite), CaSO₄-2H₂O (plaster of Paris), and Ca₃(PO₄)₂ (beta-whitlockite, a form of tricalcium phosphate, TCP), (Ca₁₀(PO₄)₆(OH)₂), and tetracalcium phosphate.

40. A method for treating a wound, surgical site, or tissue in need thereof, the method comprising administering the cell composition of any of embodiments 29 through 39 to a subject in need thereof in an amount effective to induce tissue growth or tissue regeneration.

41. A method of treating a bone injury, disorder or disease in a subject in need thereof, the method comprising administering to the subject an effective amount of the cell composition of any of embodiments 31 through 39.

42. The method of embodiment 41, wherein the bone disorder or disease is selected from the group consisting of osteoporosis, osteopenia, osteonecrosis, fracture, non-union fracture, mal-union fracture, delayed union fractures, compression fracture, maxillo-facial fractures, bone reconstruction, cranio-facial bone reconstruction, osteogenesis imperfecta, osteolytic bone cancer, Paget's Disease, endocrinological disorders, hypophsophatemia, hypocalcemia, renal osteodystrophy, osteomalacia, adynamic bone disease, rheumatoid arthritis, hyperparathyroidism, primary hyperparathyroidism, secondary hyperparathyroidism, periodontal disease, Gorham-Stout disease and McCune-Albright syndrome.

43. The method of embodiment 41 for inducing spinal fusion of vertebra comprising administering the cell composition of any one embodiments 31 through 39 between vertebra to be fused in a subject in an amount effective to induce bone growth.

44. A kit for use in the method of embodiments 41 through 43 comprising: genetically engineered minimally passaged single donor cells; and a tissue engineering scaffold, matrix, or material forming a matrix.

45. The kit of embodiment 44 wherein the cells are fibroblasts.

46. The kit of embodiment 45 wherein the cells are pluripotent or multipotent fibroblasts.

47. The kit of embodiment 44 wherein the tissue scaffold or matrix is or forms a part of a bone implant or cement.

48. The kit of embodiment 45 wherein the fibroblasts are minimally passaged autologous dermal fibroblasts.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

We claim:

1. A method of stimulating bone formation in the body, the method comprising:
(a) genetically modifying autologus cells to express bone formation stimulating proteins; and
(b) implanting said genetically modified autologus cells in a location in a body of a patient identified for bone formation to induce bone formation.

2. A method as recited in claim 1, wherein said genetic modification of autological cells comprises:
(a) culturing a sample of cells from a patient; and
(b) incubating cultured cells with an integrating or non-integrating viral vector, said vector containing genes for at least one bone stimulating protein;
wherein cells are modified to express bone formation stimulating proteins by transfection of a viral vector.

3. A method as recited in claim 2, wherein said viral vector comprises an integrating lentiviral vector.

4. A method as recited in claim 2, wherein said viral vector comprises a non-integrating adenoviral vector.

5. A method as recited in claim 1, wherein said autologus cells are cells selected from the group of cells consisting of human dermal fibroblast cells, adipose tissue cells and stem cells.

6. A method as recited in claim 1, wherein said bone formation stimulating protein is a bone morphogenic protein.

7. A method as recited in claim 1, further comprising:
associating an extracellular matrix with said genetically modified cells; and
implanting the extacellular matrix and associated cells in the body of a patient.

8. A method as recited in claim 7, wherein said extracellular matrix is selected from the group consisting of a collagen sponge, bone cement, and a collagen solution.

9. A method of bone growth stimulation in the body of a patient, the method comprising:
(a) culturing a sample of cells from a patient;
(b) genetically modifying the cultured cells to express bone growth stimulating proteins;
(c) associating the genetically modified cells with an extracellular matrix; and
(d) implanting the extracellular matrix and the cells in the body of the patient.

10. A method as recited in claim 9, wherein said bone formation stimulating protein is at least one bone morphogenic protein from the family of bone morphogenic proteins.

11. A method as recited in claim 9, wherein said bone formation stimulating protein comprises BMP-2.

12. A method as recited in claim 9, wherein said cultured cells are cells selected from the group of cells consisting of human dermal fibroblast cells, adipose tissue cells and stem cells.

13. A method as recited in claim 9, wherein said extracellular matrix is selected from the group consisting of a collagen sponge, bone cement, and a collagen solution.

14. A method as recited in claim 9, wherein said genetic modification of said cultured cells comprises:
introducing genes for at least one bone growth stimulating protein into said cultured cells with a vector to produce genetically modified cells; and
separating genetically modified cells that express bone growth stimulating proteins from cells that do not.

15. A method as recited in claim 14, wherein said vector comprises an integrating lentiviral vector.

16. A method as recited in claim 14, wherein said vector is selected from the group of vectors consisting of an adenoviral vector, a miniplasmid vector, a minicircle vector and an episomal plasmid vector.

17. A method of spinal fusion in the body of a patient, the method comprising:
(a) culturing a sample of fibroblast cells from a patient;
(b) exposing the cultured fibroblast cells to a lentiviral vector with a BMP-2 gene to produce genetically modified cultured cells expressing BMP-2;
(c) associating the genetically modified cells with an extracellular matrix; and
(d) implanting the extracellular matrix and the cells between spinal vertebrae in the body of the patient.

18. A method as recited in claim 17, wherein said extracellular matrix is selected from the group consisting of a collagen sponge, bone cement, and a collagen solution.

19. The method of claim 17, wherein said implanted cells comprise a therapeutic dose sufficient to induce bone fusion.
